(19) United States
(12) Patent Application Publication
(10) Pub. No.: US 2017/0275584 A1
(43) Pub. Date: Sep. 28, 2017

(54) Method of making an artificial micro-gland using taxis

(71) Applicants: Manuel Marquez, Midlothian, VA (US); Samantha M. Marquez, Midlothian, VA (US)

(72) Inventors: Manuel Marquez, Midlothian, VA (US); Samantha M. Marquez, Midlothian, VA (US)

(21) Appl. No.: 15/606,042

(22) Filed: May 26, 2017

Related U.S. Application Data
(63) Continuation of application No. 12/860,867, filed on Aug. 21, 2010, now abandoned.
Application No. 12/860,867 is a continuation-in-part of application No. 12/726,158, filed on Mar. 17, 2010.
(60) Provisional application No. 61/257,666, filed on Nov. 3, 2009.
(60) Provisional application No. 61/165,989, filed on Apr. 2, 2009.

(51) Int. Cl. C12N 5/00; C12N 1/26; A61K 35/74; A61K 35/742

(52) U.S. Cl. CPC C12N 5/0062; A61K 35/74; A61K 35/742; C12N 1/26; A61K 2300/00

(57) ABSTRACT

A method is used for making an artificial micro-gland by taxis. A monodisperse multiple emulsion is produced with a first fluid; a second fluid confined within the first fluid; a third fluid within the second fluid. Interfaces between the fluids permit living cells dispersed in the one of the fluids to migrate towards an adjacent fluid having a different concentration of an agent affecting the metabolic activity of the living cells. Waiting, usually about 30 minutes, allows the living cells to migrate to the interface, forming the continuous membrane. Once formed, the artificial micro-gland is removed from the remains of the emulsion. The artificial micro-gland may also be given a second layer of different cells when the emission of the cells of the artificial micro-gland is used as the agent to attract the different cells. The method may also be used to produce an artificial micro-gland within an artificial micro-gland.

### CROSS-REFERENCE TO RELATED APPLICATIONS

This application is a continuation of U.S. application Ser. No. 12/860,867 filed 21 Aug. 2010, which is a continuation-in-part of U.S. application Ser. No. 12/726,158 filed 17 Mar. 2010, which claims the benefit of U.S. provisional application 61/257,666 filed 3 Nov. 2009, and U.S. provisional application 61/165,989 filed 2 Apr. 2009, all of which are hereby incorporated in their entirety by reference herein.

### TECHNICAL FIELD

In the field of bio-affecting and body-treating compositions, a method of making an artificial gland of micro-scale with a cellular membrane and bioreactor reservoir, wherein the artificial gland is useful for biological tissue and organ repair and replacement and stem cell engineering and biotechnology applications.

### BACKGROUND ART

The artificial micro-gland was first disclosed in applicant's patent applications, referenced above. The artificial micro-gland has a shell of living cells surrounding a core or reservoir. The term “living cells” is intended to broadly encompass biological units and cells as defined the parent application. The more significant applications of the invention are currently expected to employ living cells comprising fungi, algae, fibroblasts, yeast and bacteria. The reservoir is a micro-volume bio-reactor that supports a biologically active environment. For example, it may host a medicinal component or biological activity creating helpful substances for promoting healing, vaccination, or food active ingredients. The micro-gland has potential application as a means for drug and/or cell delivery within human or other animal.

The method disclosed herein for making the micro-glands stimulates micro-gland shell growth through taxis, that is, a form of tropism, involving the stimulating the motility or migration of a cell or organism towards or away from a location to form a shell usually at a liquid/liquid interface. For simplicity herein, a cell or an organism may also be referred to as a living cell.

The prime examples of the method employ double emulsions, or emulsions of emulsions, but triple or other multiple emulsions are possible. Multiple emulsions are complex systems in which dispersed droplets contain smaller droplets inside. This is referred to as “Controllable Monodisperse Multiple Emulsions,” in a paper by Liang-Yin Chu, et al. and published 2007 Wiley-VCH Verlag GmbH & Co. KGaA, Weinheim Angew. Chem. Int. Ed. 2007, 46, 8970-8974 (Chu paper), which is hereby incorporated by reference herein. The Chu paper describes the fabrication of highly monodisperse multiple emulsion using capillary microfluidics. This is but one method of making a monodisperse multiple emulsion and any method may be used for the present invention consistent with the instructions set forth herein.

The present invention takes the process of using a monodisperse multiple emulsion a step further, using taxis, and preferably chemotaxis using chemoattractants, wherein living cells or multi-cellular organisms direct their movements according to a gradient of certain chemicals. The chemicals may be either in the same environment as the living cells or in a nearby environment where their presence can be detected by the living cells. Examples of these chemicals are oxygen for bacterial migration and carbon dioxide for algae migration. Typically, presence of these agents promoting taxis is in a higher concentration in an adjoining emulsion stimulates movement of the living cells to the interface with the other emulsion to form the continuous membrane or shell of the artificial micro-gland.

These chemicals affect metabolic activity of the living cells and so their presence in higher concentrations in an adjoining or surrounding emulsion selectively draws the cells to the interface of the emulsions. Thus, upon creation of at least one emulsion containing the living cells, wherein that emulsion is confined within at least one other emulsion having a higher concentration of a chemoattractant useful to the living cells metabolism, taxis enables movement of the living cells to the interface where they form a continuous membrane surrounding the emulsion previously containing the living cells.

Seven examples of using taxis and aqueous and oil emulsions are disclosed. These involve bacteria and algae migrating to form a shell by the presence of a higher concentration of a migrating stimulant in a surrounding emulsion. The shell is sometimes referred to as a biofilm, a biomembrane, or tissue. Generally, the living cells move to the interface within the emulsions in seeking higher concentration of the stimulant. In doing so, they form a membrane surrounding the emulsion from which they came. The invention may also be used when the surrounding emulsion has a lower concentration of a chemical that is disfavored by the living cell for metabolic activity.

### SUMMARY OF INVENTION

A method of making an artificial micro-gland by taxis is disclosed. The artificial micro-gland comprises a continuous membrane of living cells forming an enclosed volume or reservoir that serves as a bioreactor. Taxis is the self-movement of cells seeking or avoiding a metabolic stimulant or agent. The method includes a step of producing a monodisperse multiple emulsion, typically using a microfluidic device. The monodisperse multiple emulsion comprises a first fluid serving as a host environment; a second fluid confined within the host environment; a third fluid within the second fluid so that there is an interface between the second fluid and the third fluid. Living cells are dispersed in the third fluid. An agent capable of affecting the metabolic activity of the living cells is present within the second fluid at a higher concentration than in the third fluid. Waiting, usually about 30 minutes, allows the living cells to migrate to the interface between the second fluid and the third fluid to form the continuous membrane around the third fluid creating the artificial micro-gland. Once formed, the artificial micro-gland is washed or removed from the first fluid and the second fluid. In variations of the method, the living cells can be added to the first or the third fluids and the stimulant added to an adjacent fluid. The artificial micro-gland may also be given a second layer of different cells when an agent is emitted by the cells of the continuous membrane and that agent is used to attract the different cells to form the second layer. The method may also be used to produce an artificial micro-gland within an artificial micro-gland.

### Technical Problem

The fabrication of artificial micro-glands needs to be made simpler, easier, faster, and readily reproducible to accommodate their wide-spread use in the healing arts in the biomedical or biotechnological fields. Improvements are needed in enabling full control of the shell/reservoir structure of the artificial micro-gland.

### Solution to Problem

Artificial micro-glands can be made simply and more easily by using a monodisperse multiple emulsion as templates having distinct fluids with one or more interfaces between the fluids in the emulsion. The cells placed in one fluid migrate on their own to the interface to assemble and surround an inner fluid and form the artificial micro-gland.

### Advantageous Effects of Invention

Simple, easier, faster, and readily reproducible fabrication of artificial micro-glands will promote greater use of artificial micro-glands to improve tissue and organ repair, and the delivery of treatments incident to healing and recovery from cellular injuries. Additionally, the method enables greater control of the shell/reservoir structure of the artificial micro-gland.

### BRIEF DESCRIPTION OF DRAWINGS

The drawings illustrate preferred embodiments of the method of the invention. New reference numbers in FIG. 2 are given the 200 series numbers. Similarly, any new reference numbers in each succeeding drawing are given a corresponding series number beginning with the figure number.

FIG. 1 is a sectional view of the three primary transitions to an artificial micro-gland starting with water/oil/water emulsions comprising cells in the inner-most water emulsion.

FIG. 2 is a sectional view of an oil/water/oil emulsion with cells in the middle water emulsion wherein the central emulsion has a higher concentration of a taxis stimulant.

FIG. 3 is a sectional view of an oil/water/oil emulsion with cells in the middle water emulsion wherein the outer or environment emulsion has a higher concentration of taxis stimulant.

FIG. 4 is a sectional view of water emulsion containing an artificial micro-gland with a water reservoir and cells leading to an artificial micro-gland with two membranes.

FIG. 5 is a sectional view of a water emulsion containing an artificial micro-gland with an oil reservoir and cells leading to an artificial micro-gland with two membranes.

FIG. 6 is a sectional view of a water emulsion containing an artificial micro-gland with an oil-within-water reservoir and cells leading to an artificial micro-gland with two membranes.

FIG. 7 is a sectional view of the transitions from a water/oil/water emulsion to an artificial micro-gland within an artificial micro-gland.

### DESCRIPTION OF EMBODIMENTS

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized and operational changes may be made, without departing from the scope of the present invention. For example, the steps in the method of the invention may be performed in any order that results making or using the artificial micro-gland.

The embodiments of the invention described herein are alternative methods of making an artificial micro-gland (105) by taxis and more specifically by chemotaxis. As in all of the methods disclosed herein, the artificial micro-gland comprises a continuous membrane of living cells surrounding and defining an enclosed volume. The enclosed volume comprises a reservoir serving as a bioreactor. This is the same artificial micro-gland as described in the parent application, U.S. application Ser. No. 12/726,158, filed 17 Mar. 2010, which has been incorporated by reference herein.

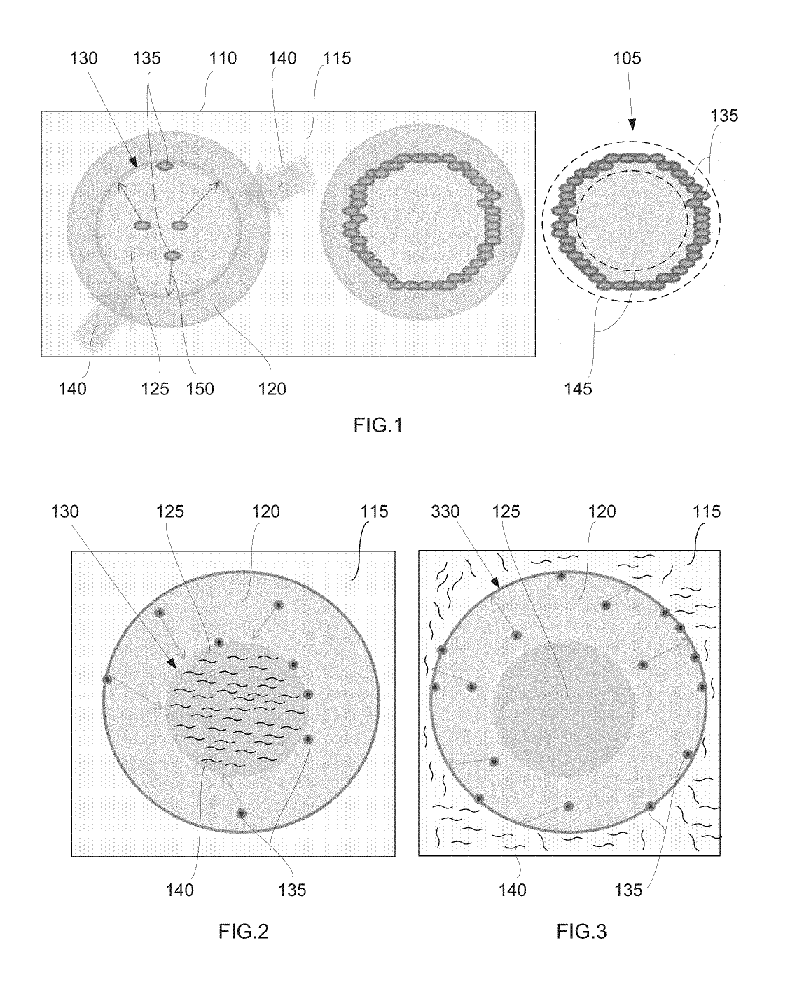

FIG. 1 illustrates a first preferred embodiment of the method of the invention. It includes a step of producing a monodisperse multiple emulsion (110) (confined within the box so designated in each of the figures). The monodisperse multiple emulsion (110) includes: a first fluid (115) serving as a host environment; a second fluid (120) confined within the host environment, the second fluid being immiscible in the first fluid (115); a third fluid (125) within the second fluid (120), the third fluid (125) being immiscible in the second fluid (120) such that there is an interface (130) between the second fluid (120) and the third fluid (125), the third fluid (125) comprising a plurality of living cells (135) dispersed therein, said living cells (135) capable of metabolic activity; and, an agent (140) (figuratively represented by the wide shaded arrows in FIG. 1) capable of affecting the metabolic activity of the living cells, the agent present within the second fluid (120) at a higher concentration than in the third fluid (125). The arrow (150) indicates the direction of movement of the living cells (135) by taxis.

The first preferred method includes a step of waiting until the living cells (135) migrate to the interface (130) between the second fluid (120) and the third fluid (125) to form the continuous membrane (145) (represented by the living cells (135) approximately between the dashed circles in the sectional view of FIG. 1) around the third fluid (125).

The first preferred method includes a step of removing the first fluid (115) and the second fluid (120) from the monodisperse multiple emulsion (110) to produce the artificial micro-gland (105).

### EXAMPLE 1

### Water/Oil/Water Emulsion Using Bacteria and Oxygen

The first preferred embodiment is illustrated with an example using a water/oil/water emulsion with bacteria as living cells and oxygen for the agent. The oxygen is a chemoattractant. A monodisperse multiple emulsion (110) is prepared using a microfluidic device. The emulsions comprise a first fluid (115), serving as a host environment, which is primarily water, but is more specifically an aqueous solution comprising Luria-Bertani broth (a common liquid medium used to grow bacteria comprising about 200 millimolar sodium chloride and 0.2 weight percent phosphorylated polyvinyl alcohol). This solution is referred to as water for simplicity of discussion. Additives to the water may be any component compatible with the living cells.

The emulsions further comprise a second fluid (120). The second fluid (120) comprises oil, or more specifically 5 centistokes silicone oil plus 2 weight percent DC749 (a common surfactant comprising cyclomethicone and trimethylsiloxylsilicate). This second fluid (120) is referred to as oil for simplicity of discussion. The oil is confined within the host environment, i.e. the water. Oil is immiscible in the first fluid (115), i.e. the water and is and semipermeable to oxygen.

The emulsions further comprise a third fluid (125). The third fluid (125) is primarily water and is more specifically an aqueous solution containing aqueous Luria-Bertani broth (˜200 millimolar sodium chloride). This third fluid (125) is referred to as water for simplicity of discussion.

The third fluid (125) is contained within the second fluid (120). The third fluid (125) is immiscible in the second fluid (120). This is the case because the water is immiscible in the oil. Immiscibility means that there is an interface (130) between the second fluid (120) and the third fluid (125) and also between the first fluid (115) and the second fluid (120). The third fluid (125), which is the water (residing inside the oil), includes a suspension of living cells (135), which in this case are bacteria, which are dispersed in the water. By definition, living cells (135) are capable of metabolic activity and this is the case for the bacteria. Examples of bacteria actually used are pseudomonas aeruginosa, b. subtilis and p. aeruginos.

The second fluid (120), which is the oil, includes an agent (140), in this case oxygen, capable of affecting the metabolic activity of the living cells (135), that is, the bacteria. The bacteria consume oxygen and discharge carbon dioxide. The presence of oxygen in a higher concentration in the oil (the second fluid) than in the water, that is than in the third fluid (125), causes the bacteria to migrate to the interface (130) between the third fluid (125) and the second fluid (120). Typically, over the course of about 10 to 30 minutes, this migration forms a continuous membrane (145) of bacteria, that is living cells (135) assemble in a biofilm to surround the third fluid (125), which is the reservoir of the artificial micro-gland (105).

Next, the monodisperse multiple emulsion (110) is poured out over a glass surface, which ruptures the emulsion, that is causes the host environment and oil to disengage from the artificial micro-gland, effectively removing the first fluid (115) and the second fluid (120) from the monodisperse multiple emulsion (110) to produce the artificial micro-gland (105). The micro-glands may be transferred to, suspended in, or preserved in another liquid, if desired.

### EXAMPLE 2

### Water/Oil/Water Emulsion Using Algae and Carbon Dioxide

The first preferred embodiment is illustrated with a second example using an water/oil/water emulsion with algae as living cells and carbon dioxide for the agent. A monodisperse multiple emulsion (110) is prepared using a microfluidic device. The emulsions comprise: a first fluid (115), serving as a host environment, which is water; a second fluid (120) comprises oil, which is confined within the host environment, i.e. the water. Oil is immiscible in the first fluid (115), i.e. the water. The third fluid (125) is water and it is contained within the second fluid (120). The water is immiscible in the oil, i.e. the second fluid (120). Immiscibility means that there is an interface (130) between the second fluid (120) and the third fluid (125) and also between the first fluid (115) and the second fluid (120). The third fluid (125), which is the water inside the oil, includes living cells (135), which in this case are algae, which are dispersed in the water. By definition, living cells (135) are capable of metabolic activity and this is the case for the algae. The second fluid (120), which is the oil, includes an agent (140), in this case carbon dioxide, capable of affecting the metabolic activity of the living cells (135), that is, the algae. The algae consume carbon dioxide and discharge oxygen in a photosynthesis process. The presence of carbon dioxide in a higher concentration in the oil (the second fluid) than in the water, that is than in the third fluid (125), causes the algae to migrate to the interface (130) between the third fluid (125) and the second fluid (120). Typically, over the course of about 10 to 30 minutes, this migration forms a continuous membrane (145) of algae, that is living cells (135) assemble to surround the third fluid (125), which is the reservoir of the artificial micro-gland (105). Next, the monodisperse multiple emulsion (110) is poured out over a glass surface, which ruptures the emulsion, that is causes the host environment and oil to disengage from the artificial micro-gland, effectively removing the first fluid (115) and the second fluid (120) from the monodisperse multiple emulsion (110) to produce the artificial micro-gland (105).

While the two examples above utilize bacteria and algae for the living cells (135), there are many other such living cells (135), which may be used and which are drawn from the panoply of eukaryotic cells and prokaryotic cells.

While the two examples above utilize oxygen and carbon dioxide as the agent (140), that is a chemoattractant, capable of affecting the metabolic activity of the living cells (135), there are many other such agents that may be utilized. Examples of such other agents are nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3), leukotine B4; lipoxins; prostaglandins; cytotaxia; f-met-leu-phe tripeptide; cytokines; kinins, cytotaxins; anaphylatoxin peptide (C5a); aspartic acid (ASP); serine (SER); and, chemo-attractants.

FIG. 2 illustrates a second preferred embodiment of the method of the invention. In this embodiment, the living cells (135) are within the second fluid (120) and migrate to the interface (130) with the third fluid (125). This second preferred embodiment similarly includes a step of producing a monodisperse multiple emulsion (110). The monodisperse multiple emulsion (110) includes: a first fluid (115) serving as a host environment; a second fluid (120) confined within the host environment, the second fluid comprising a plurality of living cells (135) dispersed therein, said living cells (135) capable of metabolic activity; a third fluid (125) within the second fluid (120), the third fluid (125) being immiscible in the second fluid (120) such that there is an interface (130) between the second fluid (120) and the third fluid (125); and, an agent (140) (figuratively represented by the squiggly lines in FIG. 2) capable of affecting the metabolic activity of the living cells (135), the agent (140) present within the third fluid (125) at a higher concentration than in the second fluid (120). In this embodiment, the first fluid (115), serving as the host environment, maintains a relatively low concentration of the agent (140) compared to the third fluid (125).

The second preferred method includes a step of waiting until the living cells (135) migrate to the interface (130) between the second fluid (120) and the third fluid (125) to form the continuous membrane (145) (represented by the living cells (135) approximately between the dashed circles in the sectional view of FIG. 1) around the third fluid (125).

The second preferred method includes a step of removing the first fluid (115) and the second fluid (120) from the monodisperse multiple emulsion (110) to produce the artificial micro-gland (105).

### EXAMPLE 3

### Oil/Water/Oil Emulsion Using Bacteria and Oxygen

The second preferred embodiment is illustrated with an example using an oil/water/oil emulsion with bacteria as living cells and oxygen for the agent. A monodisperse multiple emulsion (110) is prepared using a microfluidic device. The emulsions comprise: a first fluid (115), serving as a host environment, which is oil; a second fluid (120) comprises water, which is confined within the host environment, i,e. the oil. The third fluid (125) is a second oil and it is contained within the second fluid (120), i.e. the water. The third fluid (125), i.e. the second oil, is immiscible in the second fluid (120), i.e. the water. The second fluid (120), which is the water inside the oil or host environment, includes living cells (135), which in this case are bacteria, which are dispersed in the water, that is the second fluid (120). By definition, living cells (135) are capable of metabolic activity and this is the case for the bacteria. The second fluid (120), which is the water, includes an agent (140), in this case oxygen, capable of affecting the metabolic activity of the living cells (135), that is the bacteria. The bacteria consume oxygen and discharge carbon dioxide. The presence of oxygen in a higher concentration in the oil (the third fluid (125)) than in the water, that is than in the second fluid (120), causes the bacteria to migrate to the interface (130) between the second fluid (120) and the third fluid (125). Over the course of about 10 to 30 minutes, this migration forms a continuous membrane (145) of bacteria, that is living cells (135) surrounding the third fluid (125), which is the reservoir of the artificial micro-gland (105). Next, the monodisperse multiple emulsion (110) is poured out over a glass surface, which ruptures the emulsion, that is causes the host environment and water to disengage from the artificial micro-gland, effectively removing the first fluid (115) and the second fluid (120) from the monodisperse multiple emulsion (110) to produce the artificial micro-gland (105).

### EXAMPLE 4

### Oil/Water/Oil Emulsion Using Algae and Carbon Dioxide

The second preferred embodiment is illustrated with an example using an oil/water/oil emulsion with algae as living cells and carbon dioxide for the agent. A monodisperse multiple emulsion (110) is prepared using a microfluidic device. The emulsions comprise: a first fluid (115), serving as a host environment, which is oil; a second fluid (120) comprises algae, which is confined within the host environment, i.e. the water. The third fluid (125) is a second oil and it is contained within the second fluid (120), i.e. the water. The third fluid (125), i.e. the second oil, is immiscible in the second fluid (120), i.e. the water. The second fluid (120), which is the water inside the oil or host environment, includes living cells (135), which in this case are algae, which are dispersed in the water, that is the second fluid (120). By definition, living cells (135) are capable of metabolic activity and this is the case for the algae. The second fluid (120), which is the water, includes an agent (140), in this case carbon dioxide, capable of affecting the metabolic activity of the living cells (135), that is the algae. The algae consume carbon dioxide and discharge oxygen. The presence of carbon dioxide in a higher concentration in the oil (the third fluid (125)) than in the water, that is than in the second fluid (120), causes the algae to migrate to the interface (130) between the second fluid (120) and the third fluid (125). Over the course of about 10 to 30 minutes, this migration forms a continuous membrane (145) of algae, that is living cells (135) surrounding the third fluid (125), which is the reservoir of the artificial micro-gland (105). Next, the monodisperse multiple emulsion (110) is poured out over a glass surface, which ruptures the emulsion, that is causes the host environment and water to disengage from the artificial micro-gland, effectively removing the first fluid (115) and the second fluid (120) from the monodisperse multiple emulsion (110) to produce the artificial micro-gland (105).

Similarly to the explanation above for the first preferred embodiment, examples 3 and 4 utilize bacteria and algae for the living cells (135). Also, similarly, there are many other such living cells (135) drawn from the panoply of eukaryotic cells and prokaryotic cells.

While examples 3 and 4 utilize oxygen and carbon dioxide as the agent (140) capable of affecting the metabolic activity of the living cells (135), there are many other such agents that may be utilized. Examples of such other agents are nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins, cytotaxins; anaphylatoxin peptide (C5a); aspartic acid (ASP); serine (SER); and, chemo-attractants.

FIG. 3 illustrates a third preferred embodiment of the method of the invention. In this embodiment, the living cells (135) are within the second fluid (120) and migrate to the interface (330) with the first fluid (115). The artificial micro-gland that results has a reservoir with two distinct fluids, namely, the second fluid (120) surrounding the third fluid (125).

This third preferred embodiment similarly includes a step of producing a monodisperse multiple emulsion (110). The monodisperse multiple emulsion (110) includes: a first fluid (115) serving as a host environment; a second fluid (120) confined within the host environment, the second fluid (120), the second fluid (120) being immiscible in the first fluid (115) such that there is an interface (330) between the first fluid (115) and the second fluid (120). The second fluid (120) includes living cells (135) dispersed therein. The living cells (135) by definition are capable of metabolic activity. The monodisperse multiple emulsion (110) further includes a third fluid (125) within the second fluid (120). The monodisperse multiple emulsion (110) further includes an agent (140) (figuratively represented by the squiggly lines in FIG. 3) capable of affecting the metabolic activity of the living cells (135). The agent (140) is present within the first fluid (115) at a higher concentration than in the second fluid (120) and in the third fluid (125). Thus, the third fluid (125) maintains a relatively low concentration of the agent (140) compared to the second fluid (120) and compared to the first fluid (115).

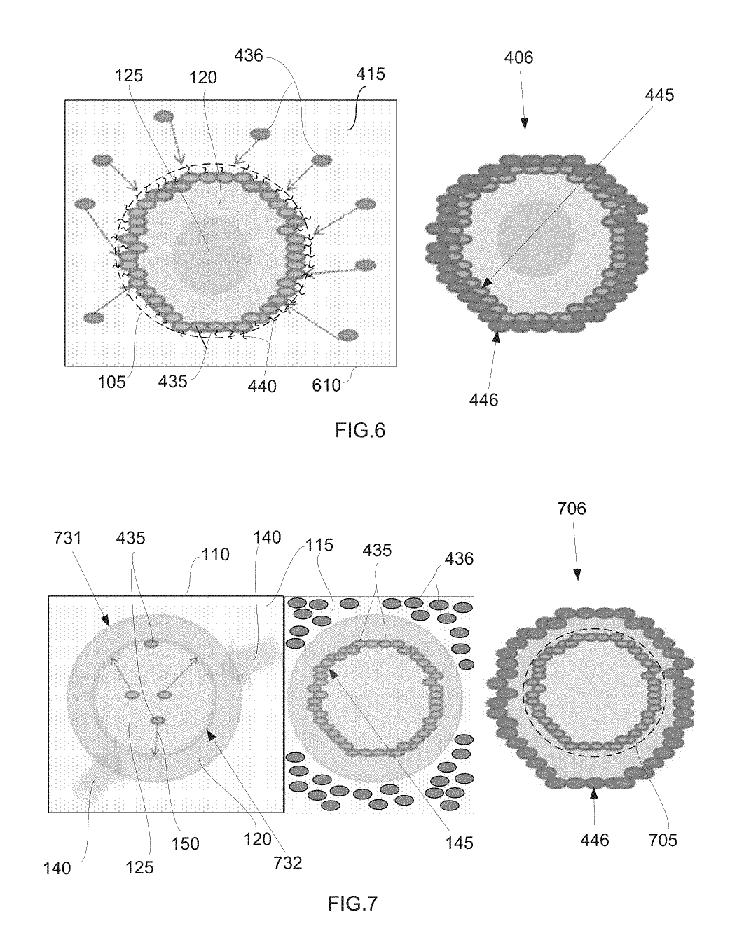

The third preferred method includes a step of waiting until the living cells (135) migrate to the interface (330) between the first fluid (115) and the second fluid (120) to form the continuous membrane (145) (represented by the living cells (135) pointed at by the arrow in FIG. 6) around the second fluid (120).

The third preferred method includes a step of removing the first fluid (115) from the monodisperse multiple emulsion (110) to produce the artificial micro-gland (105), shown in FIG. 6, having a reservoir with two distinct fluids, namely, an emulsion of the second fluid (120) surrounding the third fluid (125).

### EXAMPLE 5

### Oil/Water/Oil Emulsion Using Bacteria and Oxygen

The third preferred embodiment is illustrated with a second example using an oil/water/oil emulsion with bacteria as living cells and oxygen for the agent. A monodisperse multiple emulsion (110) is prepared using a microfluidic device. The emulsions comprise: a first fluid (115), serving as a host environment, which is oil; a second fluid (120) comprises water, which is confined within the host environment, i.e. the oil. The third fluid (125) is a second oil and it is contained within the second fluid (120), i.e. the water. The third fluid (125), i.e. the second oil, is immiscible in the second fluid (120), i.e. the water. The second fluid (120), which is the water inside the oil or host environment, includes living cells (135), which in this case are bacteria, which are dispersed in the water, that is the second fluid (120). By definition, living cells (135) are capable of metabolic activity and this is the case for the bacteria. The second fluid (120), which is the water, includes an agent (140), in this case oxygen, capable of affecting the metabolic activity of the living cells (135), that is the bacteria. The bacteria consume oxygen and discharge carbon dioxide. The presence of oxygen in a higher concentration in the oil (the first fluid (115)) than in the water, that is than in the second fluid (120), causes the bacteria to migrate to the interface (330) between the first fluid (115) and the second fluid (120). The second oil (the third fluid (125)) also contains a low concentration of oxygen. Over the course of about 10 to 30 minutes, this migration to the interface (330) forms a continuous membrane (145) of bacteria, that is living cells (135) surrounding the second fluid (120) and also the third fluid (125), which is within the second fluid (120). The continuous membrane (145), thus, has a reservoir comprising two distinct fluids, namely, an emulsion comprising the water surrounding the second oil.

Next, the monodisperse multiple emulsion (110) is poured out over a glass surface, which causes the oil to disengage from the artificial micro-gland, effectively removing the first fluid (115) from the monodisperse multiple emulsion (110) to produce the artificial micro-gland (105).

While the example used to illustrate the third preferred embodiment utilizes bacteria for the living cells (135), there are many other such living cells (135) drawn from the panoply of eukaryotic cells and prokaryotic cells that may be used. In addition to oxygen, other examples of the agent (140) capable of affecting the metabolic activity of the living cells (135) include carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, chemo-attractants.

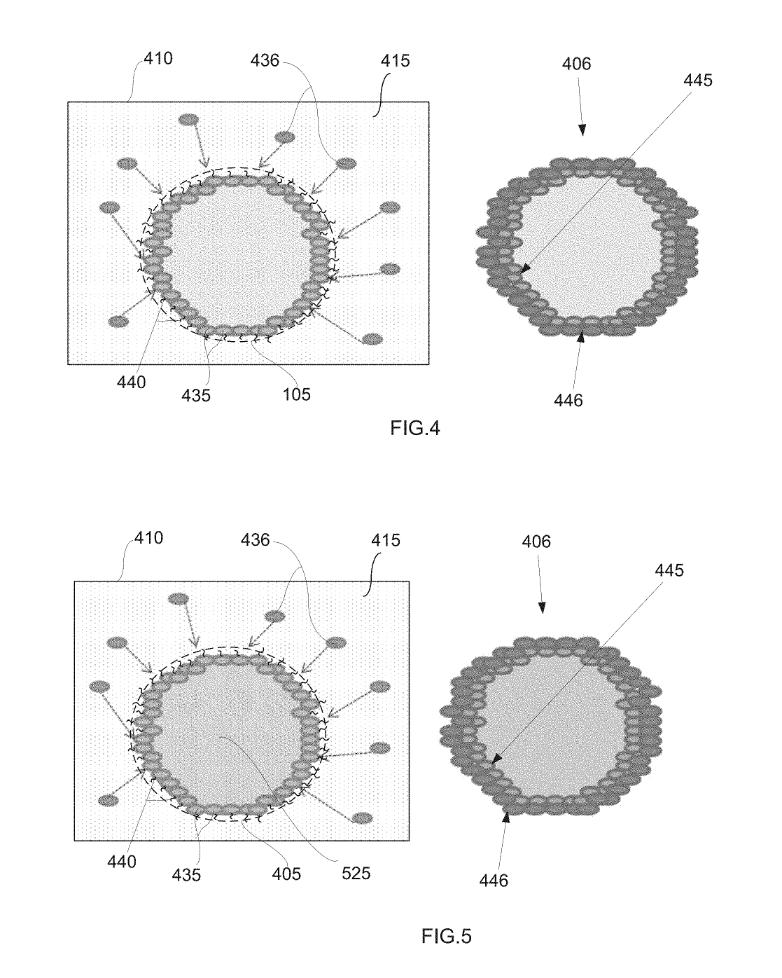

FIG. 4, FIG. 5 and FIG. 6 illustrate related preferred embodiments of the method of the invention. They are related in that each figure depicts a method of adding one or more additional shells of continuous membrane to one of the artificial micro-glands described above, or to any other artificial micro-gland. When one or more additional continuous membranes are added, the resulting artificial micro-gland is termed a multi-shell micro-gland (406), wherein the term “shell” refers to each continuous membrane. This method can take advantage of symbiotic relationships between different living cells in each membrane. For example, bacteria in one continuous membrane produce carbon dioxide from oxygen. The carbon dioxide serves the metabolic activity of algae in a second continuous membrane, which is an algal membrane or biofilm. Cooperatively, the algae produce oxygen which in turn can be used to promote the metabolic activity of the bacteria. This is an example of two cells-symbiotic-cooperation for the design and creation of complex shells-membranes in the artificial-micro-gland.

FIG. 4 illustrates a fourth preferred embodiment that is a method of making a multi-shell artificial micro-gland (406) by taxis, the multi-shell artificial micro-gland (406) comprising a plurality of continuous membranes of living cells, for example a first continuous membrane (445), also descriptively known as an inside continuous membrane, and a second continuous membrane (446), also descriptively known as an outside continuous membrane. The agent (440), or stimulant enabling taxis, is a chemical emitted by the inside continuous membrane, that is the first continuous membrane (445) of the multi-shell artificial micro-gland (406). As with all artificial micro-glands, the continuous membranes define an enclosed volume, the enclosed volume comprising a reservoir serving as a bioreactor.

The method of the fourth preferred embodiment includes a step of producing a monodisperse emulsion (410) (confined within the box so designated in FIG. 4). The monodisperse emulsion (410) includes a fluid (415) serving as a host environment.

The monodisperse emulsion (410) further includes an artificial micro-gland (105) (confined approximately within the dashed circle so designated) within the host environment. The artificial micro-gland (105) includes a first continuous membrane (445) of first living cells (435) surrounding a reservoir. Consistent with the artificial micro-glands described herein, the reservoir of the artificial micro-gland (105) may comprise oil, water, oil and water emulsion, or any other combination of liquids, gases and cells serving the bioreactor function of the reservoir. While a different reference number is used to indicate the multi-shell artificial micro-gland (406), this is done to clarify the explanation, rather than suggest that the multi-shell artificial micro-gland (406) is not simply also an artificial micro-gland as is broadly defined herein and in the parent application.

The monodisperse emulsion (410) further includes a plurality of second living cells (436) dispersed within the fluid (415). The second living cells (436) are capable of metabolic activity stimulated by discharges of an agent (440) from the first living cells (435).

The method of the fourth preferred embodiment further includes a step of waiting until the second living cells (436) migrate to the first living cells (435) to form a second continuous membrane (446) covering the first living cells (435). An exemplary waiting period is about 10 to 30 minutes. This second continuous membrane (446) is effectively an outside continuous membrane covering the first continuous membrane (445), which is effectively an inside continuous membrane. If additional shells are desired, they may be added in like manner.

The method of the fourth preferred embodiment further includes a step of removing the fluid (415) from the emulsion to produce the multi-shell artificial micro-gland (406) comprising the second continuous membrane (446) of second living cells (436).

Preferably, in the fourth preferred embodiment, the fluid (415) comprises oil or water; the first living cells (435) are either eukaryotic cells or prokaryotic cells; and, the second living cells (436) are also eukaryotic cells or prokaryotic cells, provided that the second living cells (436) are different than the first living cells (435).

### EXAMPLE 6

### Multi-Shell Artificial Micro-Gland with Reservoir of Oil

FIG. 5 illustrates the fourth preferred embodiment using an artificial micro-gland (105) (confined approximately within the dashed circle so designated) with oil (525) in its reservoir. This is the same artificial micro-gland as is described above for Example 3 of the second preferred embodiment of the method of the invention illustrated in FIG. 2.

A monodisperse emulsion (410) is produced using a microfluidic device. The monodisperse emulsion (410) comprises a fluid (415). The fluid (415) comprises water, which serves as a host environment. The monodisperse emulsion (410) further includes an artificial micro-gland (105) within the host environment. The artificial micro-gland (105) includes a first continuous membrane (445) of first living cells (435), namely bacteria, surrounding a reservoir of oil (525). The monodisperse emulsion (410) further includes a plurality of second living cells (436), namely algae, dispersed within the water, that is within the fluid (415). The second living cells (436) (the algae) are capable of metabolic activity stimulated by discharges of an agent (440) from the first living cells (435). In this case, the metabolic activity of the algae is stimulated by the discharge of carbon dioxide from the bacteria. After waiting about 30 minutes, the algae form a second continuous membrane (446) covering the bacteria. This second continuous membrane (446) is effectively an outside continuous membrane covering the first continuous membrane (445) of bacteria, which is effectively an inside continuous membrane. The multi-shell artificial micro-gland (406) is then produced by flowing the monodisperse emulsion (410) over a glass plate, which removes the water from the emulsion.

### Example 7

### Multi-Shell Artificial Micro-Gland with Reservoir of Oil within Water

FIG. 6 illustrates the fourth preferred embodiment using an artificial micro-gland (105) (confined approximately within the dashed circle so designated). The artificial micro-gland (105) includes a reservoir of a second fluid (120) comprising water surrounding a third fluid (125) comprising oil. This is essentially the same artificial micro-gland as is described for the third preferred embodiment of the method of the invention illustrated in FIG. 3.

A monodisperse emulsion (410) is produced using a microfluidic device. The monodisperse emulsion (610) includes a fluid (415). The fluid (415) comprises water, which serves as a host environment. The monodisperse emulsion (610) further includes an artificial micro-gland (105) within the host environment. The artificial micro-gland (105) includes a first continuous membrane (445) of first living cells (435), namely algae, surrounding a reservoir with two distinct fluids, namely, the second fluid (120) comprising water surrounding the third fluid (125) comprising oil (525). The monodisperse emulsion (610) further includes a plurality of second living cells (436), namely bacteria, dispersed within the water, that is within the fluid (415). The second living cells (436) (the bacteria) are capable of metabolic activity stimulated by discharges of an agent (440) from the first living cells (435). In this case, the metabolic activity of the bacteria is stimulated by the discharge of oxygen from the algae. After waiting about 30 minutes, the bacteria form a second continuous membrane (446) covering the algae. This second continuous membrane (446) is effectively an outside continuous membrane covering the first continuous membrane (445) of algae, which is effectively an inside continuous membrane. The multi-shell artificial micro-gland (406) is then produced by flowing the monodisperse emulsion (610) over a glass plate, which removes the water from the emulsion.

FIG. 7 illustrates a fifth preferred embodiment of making an artificial micro-gland by taxis. This artificial micro-gland is a dual artificial micro-gland (706). The dual artificial micro-gland (706) comprises a first artificial micro-gland (705) within a second artificial micro-gland. The second artificial micro-gland is indicated by the same reference number as the dual artificial micro-gland (706).

The fifth preferred embodiment includes a step of producing a monodisperse multiple emulsion (110). The monodisperse multiple emulsion (110) comprises a first fluid (115) serving as a host environment. The first fluid (115) preferably comprises water.

The monodisperse multiple emulsion (110) further comprises a second fluid (120) confined within the host environment. The second fluid (120) is immiscible in the first fluid (115) such that there is a first interface (731) between the first fluid (115) and the second fluid (120). The second fluid (120) preferably comprises oil.

The monodisperse multiple emulsion (110) further comprises a third fluid (125) within the second fluid (120). The third fluid (125) is immiscible in the second fluid (120) such that there is a second interface (732) between the second fluid (120) and the third fluid (125). The third fluid (125) comprises first living cells (435) dispersed therein. The first living cells (435) are capable of metabolic activity. The third fluid (125) preferably comprises water. The first living cells (435) are preferably eukaryotic cells or prokaryotic cells.

The monodisperse multiple emulsion (110) further comprises an agent (140) capable of affecting the metabolic activity of the first living cells (435). The agent (140) is present within the second fluid (120) at a higher concentration than in the third fluid (125). This higher concentration causes the first living cells (435) in the third fluid (125) to move toward the second interface (732) with second fluid (120). The agent is preferably oxygen or carbon dioxide.

The fifth preferred embodiment further includes a step of waiting until the first living cells (435) migrate to the second interface (732) between the second fluid (120) and the third fluid (125) to form the continuous membrane (145) around the third fluid (125), which then forms a first artificial micro-gland (705) within the second fluid (120). An exemplary waiting time is within about 30 minutes.

The fifth preferred embodiment further includes a step of adding second living cells (436) to the first fluid (115). Preferably, this step occurs after the continuous membrane (145) has formed. Second living cells (436) are preferably eukaryotic cells or prokaryotic cells.

The fifth preferred embodiment further includes a step of waiting until the second living cells (436) migrate to the first interface (731) between the first fluid (115) and the second fluid (120) to form a second continuous membrane (446) covering the second fluid (120) and forming the dual artificial micro-gland (706).

The fifth preferred embodiment further includes a step of removing the first fluid (115) from the monodisperse multiple emulsion (110) to produce the dual artificial micro-gland (706).

The terms “include” or “including” as used herein are not restrictive, but rather is open ended. These are intended to be equivalent to “comprise” or “comprising” and effectively mean “including, but not limited to.” The term “fluid,” as used herein may include a gas or a liquid. References herein to exemplary fluids of water or oil, may also include or contain nutrients or other additives compatible with the living cells. The water or oil are the primary components and is cited for convenience, but it should be recognized that other additives may be included therein that promote or are compatible with the living cells.

The above-described embodiments including the drawings are examples of the invention and merely provide illustrations of the invention. Other embodiments will be obvious to those skilled in the art. Thus, the scope of the invention is determined by the appended claims and their legal equivalents rather than by the examples given.

### INDUSTRIAL APPLICABILITY

The invention has application to the biomedical and biotechnological industries.

What is claimed is:

1. A method of making an artificial micro-gland by taxis, the artificial micro-gland comprising a continuous membrane of living cells, the continuous membrane defining an enclosed volume, the enclosed volume comprising a reservoir serving as a bioreactor, the method comprising the steps of:
producing a monodisperse multiple emulsion, the monodisperse multiple emulsion comprising:
a first fluid serving as a host environment;
a second fluid confined within the host environment, the second fluid being immiscible in the first fluid;
a third fluid within the second fluid, the third fluid being immiscible in the second fluid such that there is an interface between the second fluid and the third fluid, the third fluid comprising a plurality of living cells dispersed therein, said living cells capable of metabolic activity; and,
an agent capable of affecting the metabolic activity of the living cells, the agent present within the second fluid at a higher concentration than in the third fluid;
waiting until the living cells migrate to the interface between the second fluid and the third fluid to form the continuous membrane around the third fluid; and,
removing the first fluid and the second fluid from the monodisperse multiple emulsion to produce the artificial micro-gland.

2. The method of claim 1, wherein the:
first fluid of the host environment comprises water;
second fluid comprises oil;
third fluid comprises water;
agent is selected from the group consisting of: oxygen; carbon dioxide; nitrogen oxide; sugar; phosphates, nitrates, sulphates, and potassium salts; cyclic adenosine monophosphate (CAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, chemo-attractants; and,
living cells are selected from the group consisting of: eukaryotic cells; and; prokaryotic cells.

3. A method of making an artificial micro-gland by taxis, the artificial micro-gland comprising a continuous membrane of living cells, the continuous membrane defining an enclosed volume, the enclosed volume comprising a reservoir serving as a bioreactor, the method comprising the steps of:
producing a monodisperse multiple emulsion, the monodisperse multiple emulsion comprising:
a first fluid serving as a host environment;
a second fluid within the host environment, the second fluid comprising a plurality of living cells dispersed therein, said living cells capable of metabolic activity;
a third fluid within the second fluid being immiscible in the second fluid such that there is an interface between the second fluid and the third fluid;
an agent capable of affecting the metabolic activity of the living cells, the agent present within the third fluid at a higher concentration than in the second fluid;
wherein the first fluid serving as the host environment maintains a relatively low concentration of the agent compared to the third fluid; and,
waiting until the living cells to migrate to the interface between the second fluid and the third fluid to form the continuous membrane around the third fluid; and,
removing the first fluid and the second fluid from the multiple emulsion to produce the artificial micro-gland,

4. The method of claim 3, wherein the:
first fluid comprises a first oil;
second fluid comprises water;
third fluid comprises a second oil;
agent is selected from the group consisting of: oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxia; f-met-leu-phe tripeptide; cytokines; kinins,cytotaxins; anaphylatoxin peptide (C5a); aspartic acid (ASP); serine (SER); and chemo-attractants; and,
living cells are selected from the group consisting of: eukaryotic cells; and; prokaryotic cells.

5. A method of making an artificial micro-gland by taxis, the artificial micro-gland comprising a continuous membrane of living cells, the continuous membrane defining an enclosed volume, the enclosed volume comprising a reservoir serving as a bioreactor, the method comprising the steps of:
producing a monodisperse multiple emulsion, the monodisperse multiple emulsion comprising:
a first fluid serving as a host environment;
a second fluid within the host environment, the second fluid being immiscible in the first fluid such that there is an interface between the first fluid and the second fluid, the second fluid comprising a plurality of living cells dispersed therein, said living cells capable of metabolic activity;
a third fluid within the second fluid; and,
an agent capable of affecting the metabolic activity of the living cells, the agent present within the first fluid at a higher concentration than in the second fluid and in the third fluid;
waiting until the living cells migrate to the interface between the first fluid and the second fluid to form the continuous membrane around the second fluid; and,
removing the first fluid from the monodisperse multiple emulsion to produce the artificial micro-gland.

6. The method of claim 5, wherein the:
first fluid comprises a first oil;
second fluid comprises water;
third fluid comprises a second oil;
agent is selected from the group consisting of: oxygen; carbon dioxide; nitrogen oxide; sugar; phosphates, nitrates, sulphates, and potassium salts; cyclic adenosine monophosphate (cAMP); inositon phospholipid (mPIP3); actin; histamine; serotonin (5HT); plaletet acting factors (PAF); arachidonic acid metabolites; diacykglyseril (IP3); leukotine B4; lipoxins; prostaglandins; cytotaxin; f-met-leu-phe tripeptide; cytokines; kinins,cytotaxins; anaphylatoxin peptide (C5a); aspartic acid (ASP); serine (SER); and, chemo-attractants; and,
the living cells are selected from the group consisting of: eukaryotic cells; and; prokaryotic cells.

7. A method of making a multi-shell artificial micro-gland by taxis, the multi-shell artificial micro-gland comprising a plurality of continuous membranes of first living cells, the continuous membranes defining an enclosed volume, the enclosed volume comprising a reservoir serving as a bioreactor, the method comprising the steps of:
producing a monodisperse emulsion, the monodisperse emulsion comprising:
a fluid serving as a host environment;
an artificial micro-gland within the host environment, the artificial micro-gland comprising a first continuous membrane of first living cells surrounding a reservoir; and,
a plurality of second living cells dispersed within the fluid, said second living cells capable of metabolic activity stimulated by discharges of an agent from the first living cells;
waiting until the second living cells migrate to the living cells to form a second continuous membrane covering the first living cells; and,
removing the fluid from the emulsion to produce the multi-shell artificial micro-gland.

8. The method of claim 7, wherein the:
fluid comprises oil or water;
first living cells are selected from the group consisting of: eukaryotic cells; and; prokaryotic cells; and,
second living cells are selected from the group consisting of: eukaryotic cells; and; prokaryotic cells, provided that the selected second living cells are different than the first living cells.

9. A method of making an artificial micro-gland by taxis, the artificial micro-gland comprising a first artificial micro-gland within a second artificial micro-gland, the method comprising the steps of:
producing a monodisperse multiple emulsion, the monodisperse multiple emulsion comprising:
a first fluid serving as a host environment;
a second fluid confined within the host environment, the second fluid being immiscible in the first fluid such that there is a first interface between the first fluid and the second fluid;
a third fluid within the second fluid, the third fluid being immiscible in the second fluid such that there is a second interface between the second fluid and the third fluid, the third fluid comprising a plurality of first living cells dispersed therein, said first living cells capable of metabolic activity; and,
an agent capable of affecting the metabolic activity of the living cells, the agent present within the second fluid at a higher concentration than in the third fluid;
waiting until the first living cells migrate to the second interface between the second fluid and the third fluid to form a continuous membrane around the third fluid, forming a first artificial micro-gland within the second fluid;
adding second living cells to the first fluid;
waiting until the second living cells migrate to the first interface between the first fluid and the second fluid to form a second continuous membrane covering the second fluid and forming the second artificial micro-gland; and,
removing the first fluid from the monodisperse multiple emulsion to produce the artificial micro-gland comprising a first artificial micro-gland within a second artificial micro-gland.

10. The method of claim 9, wherein the:
first fluid comprises water;
second fluid comprises oil;
third fluid comprises water;
agent is selected from the group consisting of: oxygen; and, carbon dioxide;
first living cells are selected from the group consisting of: eukaryotic cells; and; prokaryotic cells; and;
second living cells are selected from the group consisting of: eukaryotic cells; and; prokaryotic cells.
